(19) United States
(12) Patent Application Publication
(10) Pub. No.: US 2025/0120568 A1
(43) Pub. Date: Apr. 17, 2025

(54) IMAGE PROCESSING APPARATUS AND ACTUATION METHOD OF IMAGE PROCESSING APPARATUS

(71) Applicant: FUJIFILM Corporation, Tokyo (JP)

(72) Inventor: Hiroki WATANABE, Kanagawa (JP)

(73) Assignee: FUJIFILM Corporation, Tokyo (JP)

(21) Appl. No.: 18/991,701

(22) Filed: Dec. 23, 2024

Related U.S. Application Data
(63) Continuation of application No. PCT/JP2023/018906, filed on May 22, 2023.

(30) Foreign Application Priority Data
Jul. 6, 2022 (JP) 2022-108947

(51) Int. Cl. A61B 1/00; A61B 1/04; A61B 1/06; G06T 7/00

(52) U.S. Cl. CPC A61B 1/000096; G06T 7/0012; A61B 1/04; A61B 1/0661; G06T 2207/10068; G06T 2207/20081; G06T 2207/30096

(57) ABSTRACT

A processor apparatus functioning as the image processing apparatus includes: a first determination unit and a second determination unit learned by using a first learning data set and a second learning data set, respectively; a processor; and a memory storing a certainty-factor determination value. The processor acquires a first light source image and a second light source image obtained by photographing an evaluation target illuminated by a first light source and a second light source, respectively, inputs the first light source image to the first determination unit, calculates a determination certainty factor from a determination result of the first determination unit, and decides whether to use the determination result of the first determination unit or to use a determination result of the second determination unit based on the determination certainty factor and the certainty-factor determination value stored in the memory.

### CROSS-REFERENCE TO RELATED APPLICATIONS

The present application is a Continuation of PCT International Application No. PCT/JP2023/018906 filed on May 22, 2023 claiming priority under 35 U.S.C § 119(a) to Japanese Patent Application No. 2022-108947 filed on Jul. 6, 2022. Each of the above applications is hereby expressly incorporated by reference, in its entirety, into the present application.

### BACKGROUND OF THE INVENTION

### 1. Field of the Invention

The present invention relates to image processing apparatuses and actuation methods of image processing apparatuses, and particularly, to a technology for evaluating an evaluation target by using a captured image of the evaluation target.

### 2. Description of the Related Art

A proposed processing system in the related art performs a detection process involving detecting a region-of-interest from an image of a detection target by using a first learned model and a second learned model obtained by training based on a first learning image and a second learning image. The first learning image is obtained by photographing a detection target by using white light, and the second learning image is obtained by photographing the detection target by using special light in a wavelength range different from that of the white light (WO2021/181564A).

The first learned model is for determining the presence/absence or the position of the region-of-interest from the image of the detection target. The second learned model is for determining the state of the region-of-interest (such as the classification of a lesion).

The processing system according to WO2021/181564A performs a process involving executing the first learned model for determining the presence/absence or the position of the region-of-interest from the image captured using the white light and a process involving executing the second learned model for determining the state of the region-of-interest from the image captured using the special light.

The processing system includes a presence determination mode and a quality determination mode. When the mode is the presence determination mode, the processing system radiates normal light as illumination light. When the mode is the quality determination mode, the processing system performs control for radiating the special light as the illumination light and switches the learned model to be used from the first learned model to the second learned model in conjunction with this switching of light sources.

In the processing system according to WO2021/181564A, the switching between the first learned model and the second learned model is performed in accordance with whether the scene is for finding a lesion or is for accurately classifying the stage of the found lesion. In detail, for example, a processing unit of the processing system switches the first learned model to the second learned model when the region-of-interest detected in the presence determination mode has a large size, when the position of the region-of-interest is near the center of the image, or when the estimated probability of the region-of-interest is greater than or equal to a predetermined threshold value. The switching between the first learned model and the second learned model may be performed based on instructional information from a user.

### SUMMARY OF THE INVENTION

In the processing system according to WO2021/181564A, one example of the switching from the first learned model to the second learned model is a case where the estimated probability of the region-of-interest is greater than or equal to the predetermined threshold value.

With regard to the switching between the learned models in this case, since the certainty in the detection of the region-of-interest using the first learned model is high, the quality determination (i.e., the classification of the type of lesion) in the region-of-interest using the second learned model can be performed favorably. In other words, the switching from the first learned model to the second learned model is not intended for enhancing the estimated probability of the region-of-interest within the image of the detection target.

WO2021/181564A describes changing control information (first control information), which is used for controlling an endoscope apparatus for increasing a first estimated probability if the first estimated probability of detection of a region-of-interest included in a first detection target image is lower than a threshold value in accordance with the learned model, to second control information, acquiring a second detection target image based on the second control information, and calculating a second estimated probability of detection of a region-of-interest included in the second detection target image in accordance with the learned model. However, the switching between learned models is not performed.

The present invention has been made in view of these circumstances, and an object thereof is to provide an image processing apparatus and an actuation method of the image processing apparatus that enable enhanced determination accuracy with respect to an evaluation target.

In order to achieve the aforementioned object, a first aspect of the invention provides an image processing apparatus including: a first determination unit and a second determination unit learned by using a first learning data set and a second learning data set obtained by photographing a target illuminated by a first light source and a second light source, respectively; a first processor; and a first memory storing a certainty-factor determination value. The first processor is configured to acquire a first light source image obtained by photographing an evaluation target illuminated by the first light source and a second light source image obtained by photographing the evaluation target illuminated by the second light source, input the first light source image to the first determination unit and calculate a determination certainty factor from a determination result of the first determination unit, and decide whether to use the determination result of the first determination unit or to use a determination result of the second determination unit based on the determination certainty factor and the certainty-factor determination value stored in the first memory.

According to the image processing apparatus according to a second aspect of the invention, in the first aspect, the first processor outputs the determination result of the first determination unit when the determination certainty factor is greater than or equal to the certainty-factor determination value, and outputs the determination result of the second determination unit when the determination certainty factor is smaller than the certainty-factor determination value.

According to the image processing apparatus according to a third aspect of the invention, in the first aspect, the certainty-factor determination value stored in the first memory includes a first certainty-factor determination value and a second certainty-factor determination value smaller than the first certainty-factor determination value. The first processor outputs the determination result of the first determination unit when the determination certainty factor is greater than or equal to the first certainty-factor determination value, and outputs the determination result of the second determination unit when the determination certainty factor is smaller than the second certainty-factor determination value.

According to the image processing apparatus according to a fourth aspect of the invention, in the first or second aspect, when the determination certainty factor is smaller than the certainty-factor determination value, the first processor acquires the second light source image in place of the first light source image, causes the second determination unit to receive the second light source image, and causes the second determination unit to output the determination result.

According to the image processing apparatus according to a fifth aspect of the invention, in any one of the first to third aspects, the first processor is configured to alternately and successively acquire the first light source image and the second light source image, and select whether to output the first light source image to the first determination unit or to output the second light source image to the second determination unit based on the determination certainty factor and the certainty-factor determination value stored in the first memory.

According to the image processing apparatus according to a sixth aspect of the invention, in any one of the first to fifth aspects, wherein the determination result output by each of the first determination unit and the second determination unit indicates a presence or an absence of a lesion, a classification of the lesion, a degree of severity of the lesion, or remission or non-remission of an inflammatory disease.

According to the image processing apparatus according to a seventh aspect of the invention, in any one of the first to sixth aspects, the learned first determination unit and the learned second determination unit include a learned first learning model and a learned second learning model that are learned by using the first learning data set and the second learning data set, respectively, and one or two second processors executing the first learning model and the second learning model.

According to the image processing apparatus according to an eighth aspect of the invention, in any one of the first to seventh aspects, the first processor acquires a plurality of determination results with respect to a plurality of regions of the first light source image from the first determination unit, and calculates the determination certainty factor based on the plurality of determination results.

According to the image processing apparatus according to a ninth aspect of the invention, in any one of the first to seventh aspects, the first processor acquires a plurality of determination results with respect to a plurality of successive first light source images from the first determination unit that successively receives the first light source images, and calculates the determination certainty factor based on the plurality of determination results.

According to the image processing apparatus according to a tenth aspect of the invention, in any one of the first to ninth aspects, illumination light from one of the first light source and the second light source is special light in a narrow band, illumination light from the other light source is white light, and each of the first light source image and the second light source image is an endoscopic image captured by an endoscope.

An eleventh aspect of the invention provides an actuation method of an image processing apparatus including a first determination unit and a second determination unit learned by using a first learning data set and a second learning data set obtained by photographing a target illuminated by a first light source and a second light source, respectively, a first processor, and a first memory storing a certainty-factor determination value. The actuation method of the image processing apparatus includes: a step for causing the first processor to acquire a first light source image obtained by photographing an evaluation target illuminated by the first light source and a second light source image obtained by photographing the evaluation target illuminated by the second light source; a step for causing the first processor to input the first light source image to the first determination unit and calculate a determination certainty factor from a determination result of the first determination unit; and a step for causing the first processor to decide whether to use the determination result of the first determination unit or to use a determination result of the second determination unit based on the determination certainty factor and the certainty-factor determination value stored in the first memory.

According to the actuation method of the image processing apparatus according to a twelfth aspect of the invention, in the eleventh aspect, the actuation method further includes a step for causing the first processor to output the determination result of the first determination unit when the determination certainty factor is greater than or equal to the certainty-factor determination value, and output the determination result of the second determination unit when the determination certainty factor is smaller than the certainty-factor determination value.

According to the actuation method of the image processing apparatus according to a thirteenth aspect of the invention, in the eleventh aspect, the certainty-factor determination value stored in the first memory includes a first certainty-factor determination value and a second certainty-factor determination value smaller than the first certainty-factor determination value. The actuation method of the image processing apparatus further includes a step for causing the first processor to output the determination result of the first determination unit when the determination certainty factor is greater than or equal to the first certainty-factor determination value, and output the determination result of the second determination unit when the determination certainty factor is smaller than the second certainty-factor determination value.

According to the actuation method of the image processing apparatus according to a fourteenth aspect of the invention, in any one of the eleventh to thirteenth aspects, the image processing apparatus further includes a third processor and a second memory storing a plurality of first evaluation data sets and a plurality of second evaluation data sets obtained by photographing the target illuminated by the first light source and the second light source, respectively. The actuation method of the image processing apparatus further includes: a step for causing the third processor to input the first evaluation data sets and the second evaluation data sets respectively to the first determination unit and the second determination unit and acquire a plurality of first determination results and a plurality of second determination results from the first determination unit and the second determination unit; a step for causing the third processor to calculate a plurality of first determination certainty factors from the plurality of first determination results; a step for causing the third processor to calculate a plurality of first determination accuracies and a plurality of second determination accuracies from the plurality of first determination results and the plurality of second determination results, respectively; and a step for causing the third processor to calculate the certainty-factor determination value based on a relationship between a determination accuracy difference and each first determination certainty factor. The determination accuracy difference indicates a difference between each first determination accuracy and each second determination accuracy at a same location of the target. The first memory stores the calculated certainty-factor determination value.

According to the actuation method of the image processing apparatus according to a fifteenth aspect of the invention, in the fourteenth aspect, the third processor linearly approximates the relationship between the determination accuracy difference and each first determination certainty factor, and calculates, as the certainty-factor determination value, the first determination certainty factor when a sign of the determination accuracy difference on a linearly approximated line is reversed.

According to the invention, the determination accuracy with respect to the evaluation target can be enhanced by appropriately deciding whether to use the determination result of either of the first determination unit and the second determination unit respectively corresponding to the first light source and the second light source.

### BRIEF DESCRIPTION OF THE DRAWINGS

FIG. 1 is a system configuration diagram of an endoscope system including a processor apparatus functioning as an image processing apparatus according to an exemplary embodiment of the invention;

FIG. 2 is a block diagram illustrating an embodiment of a hardware configuration of the processor apparatus included in the endoscope system shown in FIG. 1.

FIG. 3 is a functional block diagram illustrating a first embodiment of a main processor included in the processor apparatus shown in FIG. 2;

FIG. 4 is a block diagram illustrating another embodiment of a first determination unit and a second determination unit;

FIG. 5 schematically illustrates a learning apparatus that creates a first learning model and a second learning model;

FIG. 6 is a flowchart illustrating a first embodiment of an actuation method of the image processing apparatus according to an exemplary embodiment of the invention;

FIG. 7 is a flowchart illustrating an embodiment of a calculation method of a certainty-factor determination value;

FIG. 8 illustrates an example of a calculation method of a first determination accuracy and a second determination accuracy;

FIG. 9 is a graph related to the calculation method of the certainty-factor determination value;

FIG. 10 illustrates another example of the calculation method of the first determination accuracy and the second determination accuracy;

FIG. 11 is a flowchart illustrating a second embodiment of the actuation method of the image processing apparatus according to an exemplary embodiment of the invention;

FIG. 12 is a graph related to a calculation method of each of a first certainty-factor determination value and a second certainty-factor determination value; and

FIG. 13 illustrates other graphs related to the calculation method of the certainty-factor determination value.

### DESCRIPTION OF EXEMPLARY EMBODIMENTS

Exemplary embodiments of an image processing apparatus and an actuation method of the image processing apparatus according to the invention will be described below with reference to the appended drawings.

### System Configuration

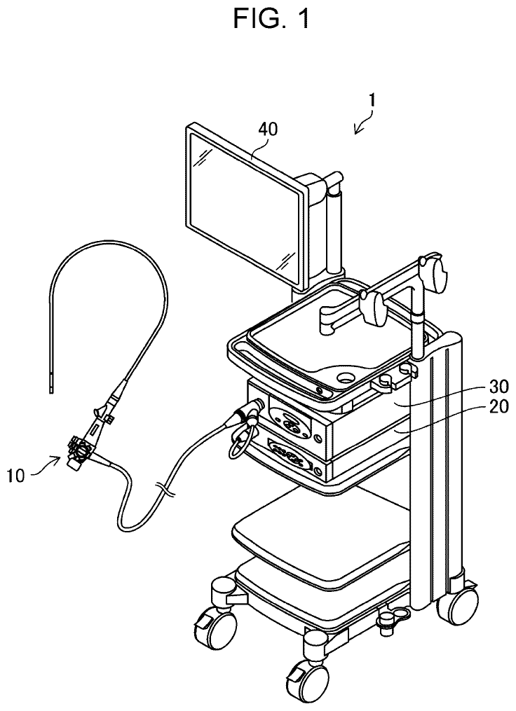

FIG. 1 is a system configuration diagram of an endoscope system including a processor apparatus functioning as the image processing apparatus according to an exemplary embodiment of the invention.

In FIG. 1, an endoscope system 1 includes an endoscope 10, a processor apparatus 20, a light source device 30, and a display device 40.

The endoscope 10 photographs an observation area within a body cavity of a subject as an evaluation target, so as to acquire an endoscopic image as a medical image. A tip portion of the endoscope 10 contains an optical system (objective lens) and an imaging element. The imaging element receives image light from the observation area via the objective lens. The imaging element converts the image light of the observation area incident on an image pick-up surface of the imaging element into an electric signal, and outputs an image signal indicating the endoscopic image.

A rear end portion of the endoscope 10 is provided with a video connector and a light guide connector for connecting the endoscope 10 to the processor apparatus 20 and the light source device 30. By attaching the video connector provided at the endoscope 10 to the processor apparatus 20, the image signal indicating the endoscopic image captured by the endoscope 10 is transmitted to the processor apparatus 20. By attaching the light guide connector provided at the endoscope 10 to the light source device 30, the illumination light emitted from the light source device 30 is radiated toward the observation area from an illumination window at the distal end surface of the endoscope 10 via the light guide connector and a light guide disposed within the endoscope 10.

The light source device 30 includes a first light source and a second light source. The light source device 30 supplies the endoscope 10, to which the light guide connector is attached, with illumination light emitted from the first light source or the second light source toward the light guide of the endoscope 10 via the light guide connector. In this example, the illumination light from one of the first light source and the second light source is special light in a narrow band, whereas the illumination light from the other light source is white light. Special light is light in a specific narrow band or light that has a peak wavelength in the specific narrow band and that is in any of various wavelength ranges according to the purpose of observation. White light is light in a wide band of white color or light in multiple wide bands with different wavelength ranges, and is also referred to as “normal light”.

The light source device 30 emits white light, special light, or white light/special light in accordance with an observation mode (e.g., a normal-light observation mode, a special-light observation mode, or a multi-frame observation mode). In the case of the multi-frame observation mode, the light source device 30 alternately emits white light and special light for every frame. The switching between white light and special light may be performed automatically, as will be described later.

When the evaluation target is illuminated with the illumination light from the first light source, the endoscope 10 photographs the illuminated evaluation target and outputs an endoscopic image as a first light source image. When the evaluation target is illuminated with the illumination light from the second light source, the endoscope 10 photographs the illuminated evaluation target and outputs an endoscopic image as a second light source image.

Specifically, the endoscope 10 captures a special light image or a white light image in accordance with the type of illumination light from the light source device 30, and outputs the captured special light image or the captured white light image. In this example, the illumination light from one of the first light source and the second light source is special light in a narrow band, whereas the illumination light from the other light source is white light.

When special light is emitted from the light source device 30, the endoscope 10 can capture, for example, a BLI (blue light imaging or blue laser imaging) image or an LCI (linked color imaging) image as a special light image. When white light is emitted from the light source device 30, the endoscope 10 can capture a white light image (WLI (white light imaging) image).

Special light for BLI is illumination light with a high proportion of violet (V) light that has high absorbance in surface-layer blood vessels and with a reduced proportion of green (G) light that has high absorbance in middle-layer blood vessels, and is suitable for generating an image (BLI image) suitable for highlighting the blood vessels and the structure of a mucous-membrane surface layer of a subject serving as an evaluation target.

Special light for LCI is illumination light with a higher proportion of V light than white light for WLI, and is suitable for capturing a fine tonal change, as compared with illumination light for WLI. An LCI image is an image having undergone color enhancement such that a red-tinted color is more red and a white-tinted color is more white with reference to the color near a mucous membrane by utilizing a red (R) component signal.

The special light in this example is special light in a narrow band having a peak wavelength in a 410-nm wavelength band. An endoscopic image captured with this special light may also be referred to as “410-nm image”.

### Processor Apparatus

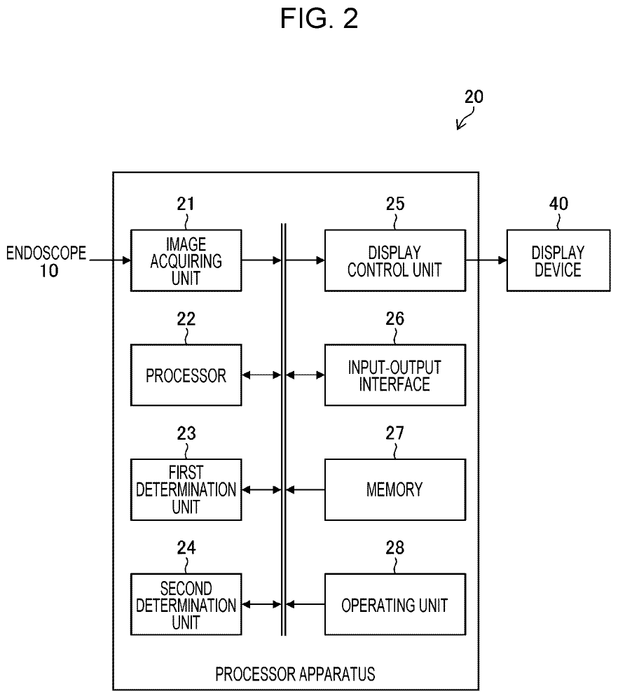

FIG. 2 is a block diagram illustrating an embodiment of a hardware configuration of the processor apparatus included in the endoscope system shown in FIG. 1.

The processor apparatus 20 shown in FIG. 2 includes an image acquiring unit 21, a processor 22 as a first processor, a first determination unit 23, a second determination unit 24, a display control unit 25, an input-output interface 26, a memory 27, and an operating unit 28.

The image acquiring unit 21 includes a connector to which the video connector of the endoscope 10 is connected, and acquires the endoscopic image captured by the imaging element disposed at the tip portion of the endoscope 10 from the endoscope 10 via the connector. The processor apparatus 20 acquires, via the connector connected to the endoscope 10, a remote signal in response to an operation performed on a hand-operable section of the endoscope 10. The remote signal includes a release signal as a command for capturing a still image.

The processor 22 includes a central processing unit (CPU) and functions as a processing unit that performs centralized control of the components of the processor apparatus 20, performs image processing on the endoscopic image acquired from the endoscope 10, performs a process for deciding whether to cause either the first determination unit 23 or the second determination unit 24 to evaluate the evaluation target, and a process for acquiring and storing the still image in response to the release signal acquired via the endoscope 10.

The first determination unit 23 or the second determination unit 24 performs an artificial intelligence (AI) process for inferring the state of the observation area of the evaluation target based on the input endoscopic image. The first determination unit 23 receives a special light image and outputs a determination result with respect to the special light image. The second determination unit 24 receives a white light image and outputs a determination result with respect to the white light image.

From the input image, each of the first determination unit 23 and the second determination unit 24 outputs a determination result indicating the presence or absence of a lesion included in the evaluation target, the classification of the lesion, the degree of severity of the lesion, or remission or non-remission of an inflammatory disease. The determination result output from each of the first determination unit 23 and the second determination unit 24 may include a determination probability (0.0 to 1.0). The first determination unit 23 and the second determination unit 24 will be described in detail later.

The display control unit 25 generates a display image based on the image-processed endoscopic image (motion picture or still image) added from the processor 22 and the determination result of the first determination unit 23 or the second determination unit 24, and outputs the display image to the display device 40.

The memory 27 includes a flash memory, a read-only memory (ROM), a random access memory (RAM), and a hard disk device. The flash memory, the ROM, or the hard disk device is a non-volatile memory that stores, for example, various programs to be executed by the processor 22. The RAM functions as a work area for processing to be executed by the processor 22, and temporarily stores, for example, a program stored in the flash memory.

The memory 27 functions as a first memory that stores a certainty-factor determination value. A certainty-factor determination value is a threshold value compared with a determination certainty factor calculated by the processor 22 and used for deciding whether to use the determination result of the first determination unit 23 or the determination result of the second determination unit 24. The certainty-factor determination value and the determination certainty factor will be described in detail later.

The memory 27 functions as a second memory that stores multiple first evaluation data sets and multiple second evaluation data sets used for calculating the certainty-factor determination value. Furthermore, the memory 27 can store a still image captured during an endoscopic diagnosis.

The display control unit 25 generates a display image based on an image-processed observation image (motion picture or still image) added from the processor 22 and an estimation result of the state of the observation area AI-processed by the processor 22, and outputs the display image to the display device 40.

The input-output interface 26 includes a connection section connected to an external device in a wired and/or wireless manner, and also includes a communication section connectable to a network. By transmitting the endoscopic image to the external device, such as a personal computer, connected via the input-output interface 26, the external device may have a partial or entire function of the image processing apparatus according to an exemplary embodiment of the invention.

The processor apparatus 20 is connected to a storage unit (not shown) via the input-output interface 26. The storage unit (not shown) is an external storage device connected to the processor apparatus 20 by, for example, a local area network (LAN), and is, for example, a file server of a system that files medical files, such as a picture archiving and communication system (PACS), or a network attached storage (NAS).

The operating unit 28 includes a power switch, a switch for manually adjusting the white balance, light quantity, and zooming, and a switch for setting the observation mode.

### First Embodiment of Processor

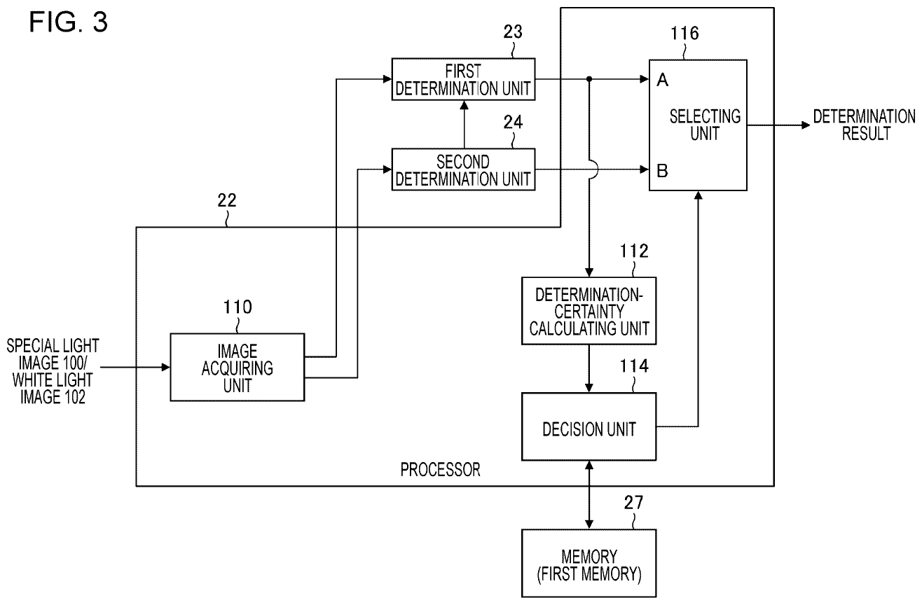

FIG. 3 is a functional block diagram illustrating a first embodiment of a main processor included in the processor apparatus shown in FIG. 2.

As shown in FIG. 3, the processor 22 includes an image acquiring unit 110, a determination-certainty calculating unit 112, a decision unit 114, and a selecting unit 116. The image acquiring unit 110 acquires a special light image 100 or a white light image 102 acquired from the endoscope 10 by the image acquiring unit 21 of the processor apparatus 20. In this example, the multi-frame observation mode is set as the observation mode. The special light image 100 and the white light image 102 are alternately captured, and the image acquiring unit 110 alternately acquires the special light image 100 and the white light image 102 for every frame.

The processor 22 inputs the special light image 100 as a first light source image to the first determination unit 23, and inputs the white light image 102 as a second light source image to the second determination unit 24.

The first determination unit 23 and the second determination unit 24 learn by using a first learning data set and a second learning data set obtained by photographing a target illuminated with the first light source (special light source) and the second light source (white light source), respectively.

The first determination unit 23 and the second determination unit 24 in this example learn to determine remission or non-remission of an inflammatory disease, and can each output information indicating “remission” or “non-remission” as a determination result and information indicating determination probabilities of “remission” and “non-remission”. For example, the information indicating the determination probability indicates a value of (0.0 to 1.0) and has a total sum of “1.0”.

The first determination unit 23 outputs a determination result with respect to the input special light image 100 to an A input terminal of the selecting unit 116 and to the determination-certainty calculating unit 112. The second determination unit 24 outputs a determination result with respect to the input white light image 102 to a B input terminal of the selecting unit 116.

The determination-certainty calculating unit 112 calculates a determination certainty factor (special-light AI determination certainty factor) based on the determination result input from the first determination unit 23.

The determination-certainty calculating unit 112 in this example calculates a special-light AI determination certainty factor (confidence_410) in accordance with Expression 1 indicated below.

$$confidence\_ 410 = \vert probability ( remission ) - probability ( non - remission ) \vert$$

In Expression 1, the probability (remission) is the determination probability of remission, and the probability (non-remission) is the determination probability of non-remission. Specifically, the determination-certainty calculating unit 112 calculates a difference between the determination probability of remission and the determination probability of non-remission from the determination result of the first determination unit 23, and calculates an absolute value of the calculated difference as the special-light AI determination certainty factor.

The special-light AI determination certainty factor (confidence_410) calculated by the determination-certainty calculating unit 112 is output to the decision unit 114.

A certainty-factor determination value is added from the memory 27 functioning as a first memory to another input terminal of the decision unit 114. Based on the special-light AI determination certainty factor (confidence_410) and the certainty-factor determination value, the decision unit 114 decides whether to use the determination result of the first determination unit 23 or the determination result of the second determination unit 24. The processor 22 (decision unit 114) according to the first embodiment decides to output the determination result of the first determination unit 23 when the special-light AI determination certainty factor (confidence_410) is greater than or equal to the certainty-factor determination value, and to output the determination result of the second determination unit 24 when the special-light AI determination certainty factor (confidence_410) is smaller than the certainty-factor determination value.

The selecting unit 116 receives the decision result of the decision unit 114 as switching information. The selecting unit 116 selects and outputs the determination result of the first determination unit 23 input to the A input terminal when the selecting unit 116 receives switching information for outputting the determination result of the first determination unit 23, and selects and outputs the determination result of the second determination unit 24 input to the B input terminal when the selecting unit 116 receives switching information for outputting the determination result of the second determination unit 24.

Accordingly, a determination result using an image with the higher determination accuracy with respect to the evaluation target between the special light image and the white light image can be acquired.

### Another Embodiment of First Determination Unit and Second Determination Unit

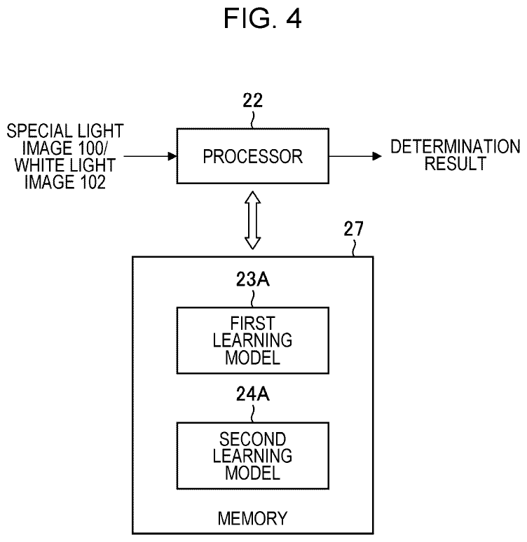

FIG. 4 is a block diagram illustrating another embodiment of the first determination unit and the second determination unit.

The processor apparatus 20 shown in FIG. 2 includes the first determination unit 23 and the second determination unit 24 separately from the processor 22. In the embodiment shown in FIG. 4, the processor 22 functions as parts of the first determination unit 23 and the second determination unit 24.

Specifically, the memory 27 stores a learned first learning model 23A and a learned second learning model 24A. The first learning model 23A is learned by using the first learning data set obtained by photographing the target illuminated by the special light source as the first light source, and the second learning model 24A is learned by using the second learning data set obtained by photographing the target illuminated by the white light source as the second light source.

The processor 22 functions as the first determination unit 23 by reading out the first learning model 23A from the memory 27 and executing the process of the first learning model 23A on the input special light image 100, and functions as the second determination unit 24 by reading out the second learning model 24A from the memory 27 and executing the process of the second learning model 24A on the input white light image 102.

As an alternative to the processor 22, one or two separate processors (second processors) executing the first learning model 23A and the second learning model 24A may be used.

### Learning Models

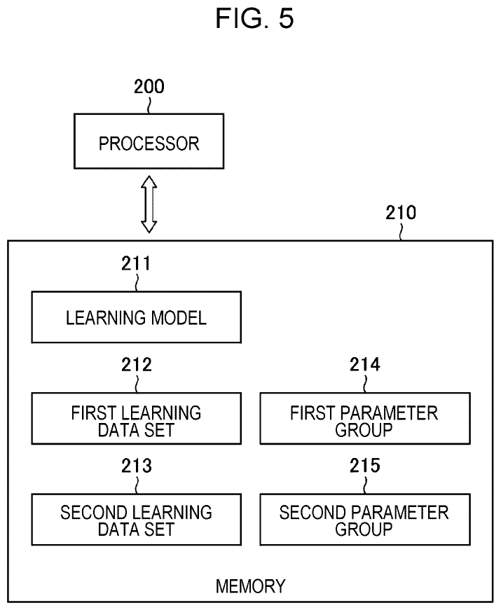

FIG. 5 schematically illustrates a learning apparatus that creates the first learning model and the second learning model.

The learning apparatus includes a processor 200 and a memory 210.

The memory 210 stores a learning model 211, a first learning data set 212, a second learning data set 213, a first parameter group 214, and a second parameter group 215.

The learning model 211 used may be a convolutional neural network (CNN) as one of general-purpose learning models. The CNN has a multilayer structure including multiple convolutional layers and multiple pooling layers. Each of the first parameter group 214 and the second parameter group 215 includes, for example, a filter coefficient of a filter called a kernel used in convolutional computing in the convolutional layers of the CNN and a weight connecting units in each layer to each other, and is set to an initial value prior to learning.

Each of the first learning data set 212 and the second learning data set 213 is a data set having N sets, each with two captured images, namely, a special light image and a white light image, of the same location, and having ground truth data attached to each set. Therefore, the first learning data set 212 is a data set constituted of N special light images and ground truth data corresponding thereto, and the second learning data set 213 is a data set constituted of N white light images and ground truth data corresponding thereto. In the case of an inflammatory bowel disease, the ground truth data can be acquired by allowing the doctor to visually inspect an endoscopic image for each region and grade the endoscopic degree of severity, or to perform a biopsy for each region and grade the pathologic degree of severity. The first learning data set 212 and the second learning data set 213 are preferably acquired from a large number of patients.

When the processor 200 performs learning of the first learning model 23A, the processor 200 executes the process of the learning model 211 using the special light images constituting the first learning data set 212 as input images, and calculates an error (loss value) among output data, the special light images, and a pair of ground truth data. Examples of the method for calculating the loss value include softmax cross entropy and sigmoid. Based on the calculated loss value, the first parameter group 214 is adjusted in accordance with error backpropagation. Error backpropagation involves causing the error to be back-propagated starting from the final layer, performing stochastic gradient decent in each layer, and repeatedly updating the first parameter group 214 until the error settles. By performing this using all of the N first learning data sets, the first parameter group 214 is optimized.

Likewise, when the processor 200 performs learning of a second learning model 24A, the processor 200 uses the second learning data set 213 to optimize the second parameter group 215.

The first learning model 23A shown in FIG. 4 is constituted of the learning model 211 and the optimized first parameter group 214, and the second learning model 24A is constituted of the learning model 211 and the optimized second parameter group 215. Therefore, in addition to the general-purpose learning model 211, the first parameter group 214 and the second parameter group 215 optimized by the learning apparatus shown in FIG. 5 are stored in the memory 27, so that the first learning model 23A and the second learning model 24A are substantially stored therein.

### First Embodiment of Actuation Method of Image Processing Apparatus

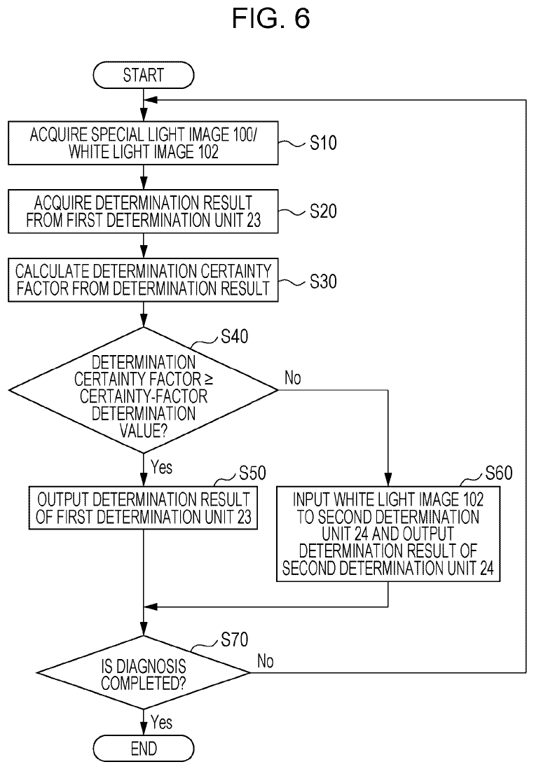

FIG. 6 is a flowchart illustrating a first embodiment of the actuation method of the image processing apparatus according to an exemplary embodiment of the invention.

The actuation method of the image processing apparatus according to the first embodiment is, for example, a method for actuating the image processing apparatus including the first determination unit 23, the second determination unit 24, the processor 22 as a first processor, and the memory 27 as a first memory shown in FIG. 3. The processor 22 executes various processes indicated below in accordance with the flowchart shown in FIG. 6.

In FIG. 6, the processor 22 alternately acquires the special light image 100 and the white light image 102 captured by the endoscope 10 during the multi-frame observation mode (step S10). As an alternative to the special light image 100 and the white light image 102 being alternately acquired for every frame in step S10, for example, the special light image 100 and the white light image 102 may be alternately acquired for every two frames.

The special light image 100 is input to the first determination unit 23, and a determination result is output from the first determination unit 23. The processor 22 acquires the determination result from the first determination unit 23 (step S20), and calculates a determination certainty factor from the acquired determination result (step S30). The determination certainty factor (special-light AI determination certainty factor (confidence_410)) can be calculated in accordance with Expression 1 indicated above.

The processor 22 compares the special-light AI determination certainty factor (confidence_410) calculated in step S30 with the certainty-factor determination value stored in the memory 27 (step S40). If the special-light AI determination certainty factor (confidence_410) is greater than or equal to the certainty-factor determination value (determination certainty factor≥certainty-factor determination value), the processor 22 proceeds to step S50 and outputs the determination result of the first determination unit 23. If the special-light AI determination certainty factor (confidence_410) is smaller than the certainty-factor determination value (determination certainty factor<certainty-factor determination value), the processor 22 proceeds to step S60 and outputs the determination result of the second determination unit 24 to which the white light image 102 is input.

Accordingly, a determination result using an image with the higher determination accuracy with respect to the evaluation target between the special light image and the white light image can be selected and output.

Subsequently, the processor 22 determines whether or not to terminate the endoscopic-image-based diagnosis in accordance with, for example, an operation performed on the operating unit 28 by the user (step S70). If the processor 22 determines that the diagnosis is not to be terminated, the processor 22 proceeds to step S10 to repeatedly execute the process from step S10 to step S70. When the processor 22 determines that the diagnosis is to be terminated, the processor 22 ends the process.

The determination result output in step S50 or step S60 is displayed on the display device 40 together with the endoscopic image, thereby assisting in the endoscopic-image-based diagnosis by the user.

### Calculation Method of Certainty-Factor Determination Value

Next, a calculation method of the certainty-factor determination value to be stored in the memory 27 will be described.

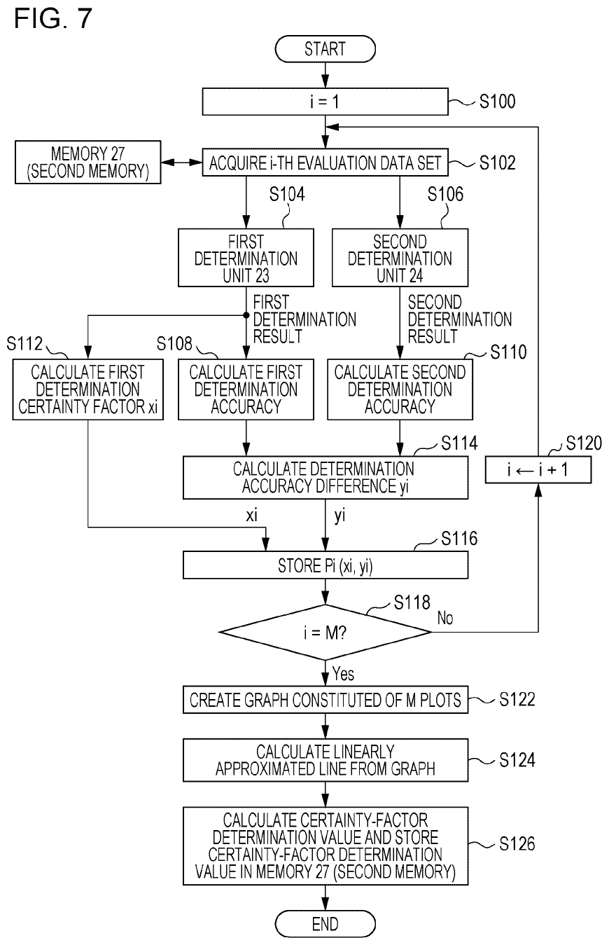

FIG. 7 is a flowchart illustrating an embodiment of the calculation method of the certainty-factor determination value.

The calculation method of the certainty-factor determination value is included in the actuation method of the image processing apparatus according to an exemplary embodiment of the invention. The image processing apparatus further includes a third processor and a second memory.

The third processor may be the same as the processor 22 functioning as the first processor, or may be a different processor. The following description relates to a case where the processor 22 functions as the third processor.

The second memory stores the multiple first evaluation data sets and the multiple second evaluation data sets obtained by photographing the target illuminated with the first light source and the second light source, respectively. In this example, the memory 27 functions as the second memory.

The multiple first evaluation data sets and the multiple second evaluation data sets stored in the memory 27 are different from the first learning data set and the second learning data set used in the learning of the first determination unit 23 and the second determination unit 24. Each of the first evaluation data sets and the second evaluation data sets is a data set having M sets, each with two captured images, namely, a special light image and a white light image, of the same location, and having ground truth data attached to each set. The first learning data set and the second learning data set may partially be used as the first evaluation data sets and the second evaluation data sets.

In FIG. 7, when the processor 22 functioning as the third processor calculates a certainty-factor determination value, the processor 22 sets a parameter i indicating one data set of the first evaluation data sets and the second evaluation data sets to 1 (step S100).

Then, the processor 22 acquires one evaluation data set of an i-th special light image and an i-th white light image from the memory 27 based on the parameter i (step S102). The processor 22 inputs the special light image in the acquired evaluation data set to the first determination unit 23, and acquires a determination result (first determination result) from the first determination unit 23 (step S104). Likewise, the processor 22 inputs the white light image in the set to the second determination unit 24, and acquires a determination result (second determination result) from the second determination unit 24 (step S106).

Subsequently, the processor 22 calculates a first determination accuracy (410-nm AI determination accuracy) from the first determination result acquired in step S104 (step S108), and calculates a second determination accuracy (WLI AI determination accuracy) from the second determination result acquired in step S106 (step S110).

An example of the calculation method of the first determination accuracy and the second determination accuracy in step S108 and step S110 will be described.

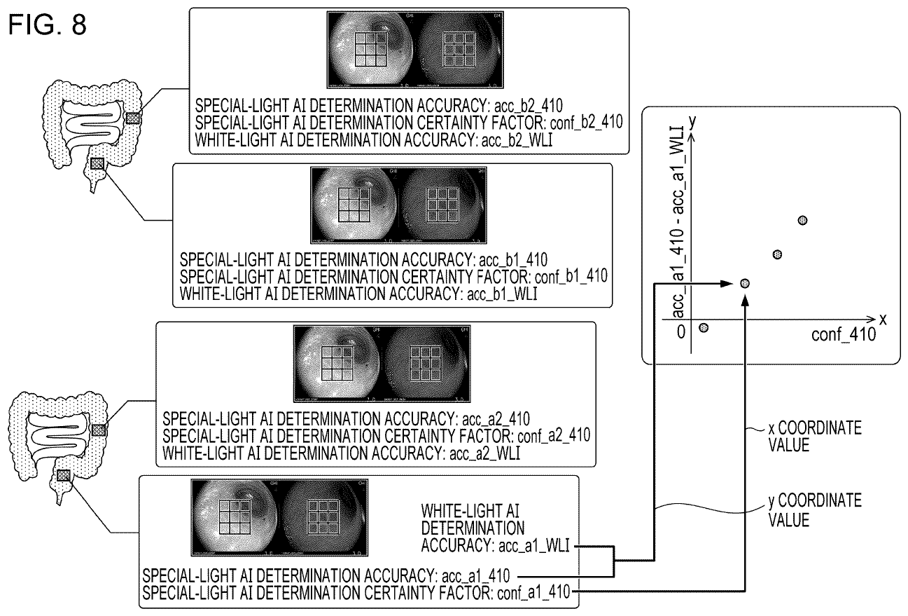

FIG. 8 illustrates an example of the calculation method of the first determination accuracy and the second determination accuracy.

FIG. 8 illustrates, as parts of the first evaluation data sets and the second evaluation data sets, four sets of special light images (a1_410, a2_410, b1_410, and b2_410) and white light images (a1_WLI, a2_WLI, b1_WLI, and b2_WLI) captured at four locations (a1, a2, b1, and b2) of the large intestine.

In step S108 and step S110, multiple determination results indicating “remission” or “non-remission” of an inflammatory disease in multiple regions (nine 3×3-split regions) of the special light image, as shown in FIG. 8, are acquired as the first determination result obtained by the first determination unit 23 and the second determination result obtained by the second determination unit 24 with respect to one set of two images, namely, the special light image (i_410) and the white light image (i_WLI) captured at the same location (i) of the large intestine. The determination of whether each of the nine regions is “remission” or “non-remission” can be performed by determining whether or not the determination probability (0.0 to 1.0) of the region is greater than or equal to 0.5.

In step S108, for example, if nine determination results (first determination results) in the nine regions of the special light image (i_410) are all correct with respect to the ground truth data, accuracy_i_410= 9/9is calculated as the first determination accuracy (410-nm AI determination accuracy) with respect to the special light image (i_410). Likewise, for example, in step S110, if three of nine determination results (second determination results) in the nine regions of the white light image (i_WLI) are correct with respect to the ground truth data, accuracy_i_WLI= 3/9 is calculated as the second determination accuracy (WLI AI determination accuracy) with respect to the white light image (i_WLI).

Referring back to FIG. 7, the processor 22 calculates a first determination certainty factor xi from the first determination result acquired in step S104 in accordance with Expression 2 indicated below (step S112).

$$xi = confidence i 410 = \frac{\begin{matrix}{\sum }_{n = 1}^{n = 9} \vert probability\_n ( remission ) - \\ probability\_n ( non - remission ) \vert\end{matrix}}{9}$$

In Expression 2, the probability (remission) is the determination probability of remission, whereas the probability (non-remission) is the determination probability of non-remission. The numerical value of 9 is the number of multiple regions for which the determination probability is determined in one special light image, as shown in FIG. 8.

Specifically, in step S112, a difference between the determination probability of remission and the determination probability of non-remission in the nine regions of the special light image is calculated from the determination results of the first determination unit 23, and an average value of nine absolute values of the calculated difference is calculated as a first determination certainty factor xi.

In accordance with Expression 3 indicated below, the processor 22 calculates a determination accuracy difference yi as a difference between the 410-nm AI determination accuracy (accuracy_i_410) as the first determination accuracy and the WLI AI determination accuracy (accuracy_i_WLI) as the second determination accuracy calculated in step S108 and step S110 (step S114).

$$yi = accuracy\_i \_ 410 - accuracy\_i \_WLI$$

The processor 22 stores, in the memory 27, a point Pi (xi, yi) having the first determination certainty factor xi calculated in step S112 as an x coordinate value and the determination accuracy difference yi calculated in step S114 as a coordinate value (step S116).

Then, the processor 22 determines whether or not the parameter i is equal to M (step S118). If i≠M (i.e., if “No”), the processor 22 increments the parameter i by 1 (step S120), and returns to step S102.

If i=M (i.e., if “Yes”), the processor 22 proceeds to step S122. In this case, since the process from step S102 to step S118 has been repeated for M times (multiple times), M number of first determination certainty factors xi are acquired. Likewise, M first determination accuracies (410-nm AI determination accuracies) are calculated from M first determination results, M second determination accuracies (WLI AI determination accuracies) are calculated from M second determination results, and M determination accuracy differences yi indicating differences between the first determination accuracies and the second determination accuracies are acquired. As a result, M points Pi (i=1 to M) are stored in the memory 27. In step S122, a graph constituted of M plots is created.

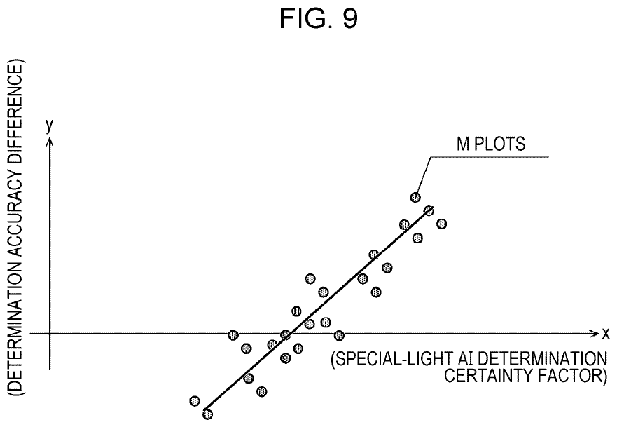

FIG. 9 is a graph related to the calculation method of the certainty-factor determination value.

FIG. 9 illustrates an example of the graph in which M points are plotted. As a result of studying ulcerative colitis by using a 410-nm image as a special light image, the graph in FIG. 9 is obtained.

In FIG. 9, the horizontal axis (x axis) indicates the special-light AI determination certainty factor, whereas the vertical axis (y axis) indicates the determination accuracy difference.

It is apparent from FIG. 9 that the 410-nm AI determination accuracy becomes higher than the WLI AI determination accuracy as the special-light AI determination certainty factor increases, and that the WLI AI determination accuracy becomes higher than the 410-nm AI determination accuracy as the special-light AI determination certainty factor decreases. Specifically, it is found that the determination accuracy difference becomes linear relative to the special-light AI determination certainty factor. In other words, the scenes that a WLI image and a 410-nm image excel in determination are different from each other. In a scene where the 410-nm image excels in determination, the WLI image does not excel in the determination. In contrast, in a scene where the WLI image excels in determination, the 410-nm image does not excel in the determination.

This is established not only in ulcerative colitis but also in any disease so long as the two light sources have different wavelength configurations and the capturing characteristics are different.

The processor 22 linearly approximates the relationship between the determination accuracy difference and the first determination certainty factor, calculates, as the certainty-factor determination value, the first determination certainty factor (i.e., an x coordinate value of a point where the linearly approximated line intersects the x axis) when the sign of the determination accuracy difference on the linearly approximated line is reversed, and stores the first determination certainty factor in the memory 27 (step S126).

For example, if the certainty-factor determination value calculated as described above and stored in the memory 27 is 0.8, when the determination certainty factor calculated from the special-light-image determination result of the first determination unit 23 becomes smaller than 0.8, the white-light-image determination result of the second determination unit 24 has higher determination accuracy. Therefore, it is preferable to switch from the determination output of the first determination unit 23 to the determination output of the second determination unit 24.

Specifically, as shown in FIG. 9, the determination accuracy difference becomes linear relative to the special-light AI determination certainty factor. Thus, by utilizing this linear relationship and referring to the special-light AI determination certainty factor during a diagnosis, it is possible to sense which light source is suitable for determining the current scene. In other words, the special-light AI determination certainty factor is calculated during a diagnosis, and the 410-nm AI determination accuracy is determined to have higher diagnostic accuracy (higher scene adequacy) than the WLI AI determination accuracy if the special-light AI determination certainty factor is greater than or equal to the certainty-factor determination value. In contrast, if the special-light AI determination certainty factor is smaller than the certainty-factor determination value, it can be determined that the WLI AI determination accuracy has higher diagnostic accuracy than the 410-nm AI determination accuracy. This enables appropriate selection and output between the determination result of the first determination unit 23 and the determination result of the second determination unit 24.

### Reasons Why Scene Suitable for Determination Varies For Each Light Source

### Disease Type

A WLI image is suitable for determining the degree of severity based on redness in a mucous membrane caused by, for example, an inflammatory disease, whereas a special light image is suitable for determining the degree of severity based on a vascular state in a polyp or an irregularity state of a mucous membrane caused by, for example, a neoplastic disease.

### Image Capturing Condition

Because the states of surface-layer blood vessels and a mucous membrane can be ascertained better with a special light image than with a WLI image, a diagnosis using a special light image is suitable in a relatively close-view image where these feature quantities appear clearly. However, since a special light image is normally darker than a WLI image, the brighter WLI image provides better viewability of the states of the blood vessels and the mucous membrane and is thus suitable for a diagnosis in a far-view image.

### Degree of Severity

For example, in an inflammatory disease, redness in a mucous membrane is low in a clearly mild case, and the redness in the mucous membrane is low in a clearly severe case. Therefore, when detecting and differentiating clearly mild and severe ranges, a diagnosis based on a WLI image is suitable since the redness in the mucous membrane can be ascertained well. However, in a moderate range, the redness in the mucous membrane is not uniform, and the state of the surface-layer blood vessels or the degree of bleeding has a better correlation with the degree of severity within the moderate range than the redness in the mucous membrane. Therefore, a special light image is more suitable for detecting and differentiating a moderate range than a WLI image.

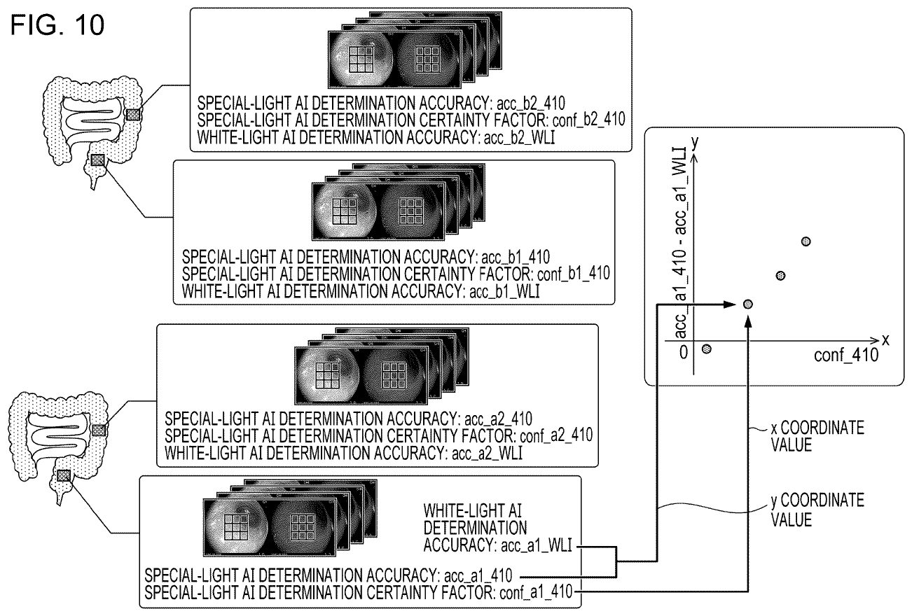

FIG. 10 illustrates another example of the calculation method of the first determination accuracy and the second determination accuracy.

FIG. 10 illustrates, as parts of the first evaluation data sets and the second evaluation data sets, four sets multiplied by multiple special light images (a1_410, a2_410, b1_410, and b2_410) and multiple white light images (a1_WLI, a2_WLI, b1_WLI, and b2_WLI) captured multiple times at the four locations (a1, a2, b1, and b2) of the large intestine. The multiple images may be the number of frames captured in one second.

In this case, with regard to multiple special light images (i_410) captured at the same location (i) of the large intestine, first determination accuracies accuracy_i_410 described with reference to FIG. 8 are calculated, and an average value of the multiple calculated first determination accuracies accuracy_i_410 can be set as a first determination accuracy average (accuracy_i_410).

Likewise, with regard to multiple white light images (i_WLI), second determination accuracies accuracy_i_WLI described with reference to FIG. 8 are calculated, and an average value of the multiple calculated second determination accuracies accuracy_i_WLI can be set as a second determination accuracy average (accuracy_i_WLI).

In this case, the determination accuracy difference yi is an average determination accuracy difference as a difference between the first determination accuracy average (accuracy_i_410) and the second determination accuracy average (accuracy_i_WLI).

On the other hand, the first determination certainty factor xi may be set as an average determination certainty factor average (confidence_i_410) by calculating multiple images' worth of first determination certainty factors in accordance with Expression 2 and averaging out the calculated multiple images' worth of first determination certainty factors.

By creating a graph, as shown in FIG. 9, using the average determination accuracy difference and the average determination certainty factor of multiple successive images for increasing the robustness instead of in units of images as shown in FIG. 8, the certainty-factor determination value can be determined.

In step S30 in FIG. 6, the determination certainty factor (special-light AI determination certainty factor (confidence_410)) is calculated in accordance with Expression 1. Alternatively, the determination certainty factor (special-light AI determination certainty factor (confidence_410)) may be calculated in accordance with Expression 2 or may be calculated as an average determination certainty factor of multiple images.

### Second Embodiment of Actuation Method of Image Processing Apparatus

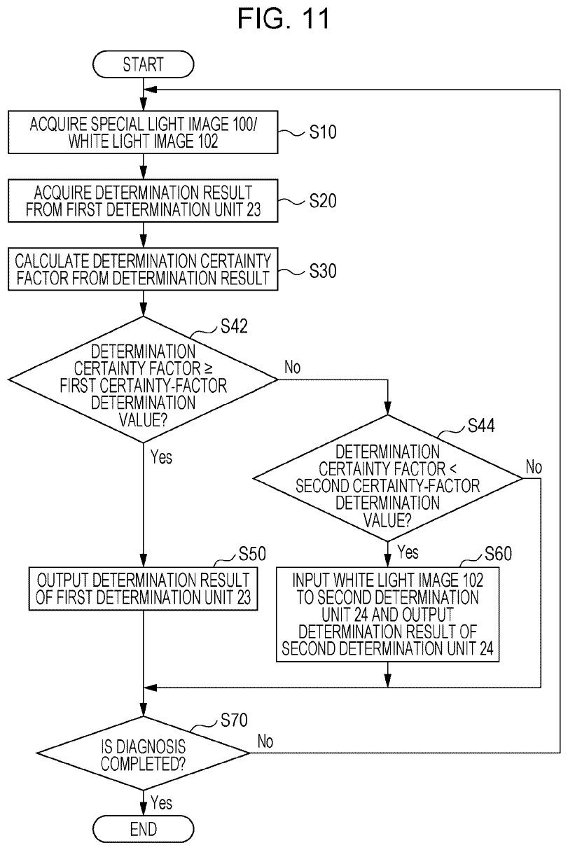

FIG. 11 is a flowchart illustrating a second embodiment of the actuation method of the image processing apparatus according to an exemplary embodiment of the invention.

In FIG. 11, steps identical to those in the actuation method of the image processing apparatus according to the first embodiment shown in FIG. 6 are given the same step numbers, and detailed descriptions thereof will be omitted.

The actuation method of the image processing apparatus according to the second embodiment is, for example, a method for actuating the image processing apparatus including the first determination unit 23, the second determination unit 24, the processor 22 as a first processor, and the memory 27 as a first memory shown in FIG. 3. The memory 27 stores a first certainty-factor determination value as a certainty-factor determination value, and also stores a second certainty-factor determination value smaller than the first certainty-factor determination value.

The actuation method of the image processing apparatus according to the second embodiment shown in FIG. 11 is different from the actuation method of the image processing apparatus according to the first embodiment in that step S42 and step S44 are performed in place of step S40 shown in FIG. 6.

The processor 22 compares the determination certainty factor (special-light AI determination certainty factor (confidence_410)) calculated in step S30 with the first certainty-factor determination value stored in the memory 27 (step S42). If the special-light AI determination certainty factor (confidence_410) is greater than or equal to the first certainty-factor determination value (determination certainty factor≥first certainty-factor determination value), the processor 22 proceeds to step S50 and outputs the determination result of the first determination unit 23. If the special-light AI determination certainty factor (confidence_410) is smaller than the first certainty-factor determination value (determination certainty factor<first certainty-factor determination value), the processor 22 proceeds to step S44.

In step S44, the processor 22 further compares the special-light AI determination certainty factor (confidence_410) with the second certainty-factor determination value stored in the memory 27. If the special-light AI determination certainty factor (confidence_410) is smaller than the second certainty-factor determination value (determination certainty factor<second certainty-factor determination value), the processor 22 proceeds to step S60 and outputs the determination result of the second determination unit 24 to which the white light image 102 is input.

On the other hand, if the special-light AI determination certainty factor (confidence_410) is greater than or equal to the second certainty-factor determination value (i.e., second certainty-factor determination value≤determination certainty factor<first certainty-factor determination value) in step S44, the processor 22 proceeds to step S70 without outputting either of the determination result of the first determination unit 23 and the determination result of the second determination unit 24.

Next, the first certainty-factor determination value and the second certainty-factor determination value will be described.

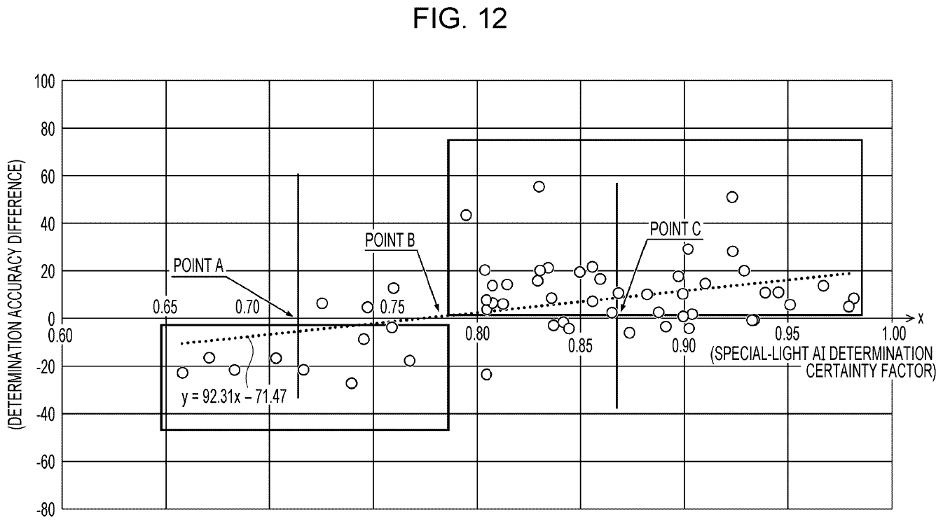

FIG. 12 is a graph related to a calculation method of each of the first certainty-factor determination value and the second certainty-factor determination value.

The graph shown in FIG. 12 is another example of the graph in which M points are plotted. The M points are calculated based on M first evaluation data sets and M second evaluation data sets.

A calculation method of M points Pi (xi, yi) (i=1 to M) can be performed similarly to the method described with reference to FIG. 7.

The processor 22 calculates a linearly approximated line from the graph shown in FIG. 12. The linearly approximated line in this example indicates that y=92.31x−71.47, as indicated with a dotted line in FIG. 12. In this case, point B where the linearly approximated line intersects the x axis is 0.77. In the calculation method of the certainty-factor determination value shown in FIG. 7, 0.77 is stored as the certainty-factor determination value in the memory 27.

The actuation method of the image processing apparatus according to the second embodiment involves causing the memory 27 to store the certainty-factor determination value (first certainty-factor determination value) at point C, which is larger than that at point B, and the certainty-factor determination value (second certainty-factor determination value) at point A, which is smaller than that at point B, in place of the certainty-factor determination value indicated at point B shown in FIG. 12, and using the first certainty-factor determination value and the second certainty-factor determination value as threshold values.

The first certainty-factor determination value at point C may be set as an x coordinate value that is α % of a maximum value yₘₐₓ of the linearly approximated line within a range surrounding a positive point Pi shown in FIG. 12, and the second certainty-factor determination value at point A may be set as an x coordinate value that is β % of a minimum value yₘᵢₙ of the linearly approximated line within a range surrounding a negative point Pi shown in FIG. 12. Likewise, the second certainty-factor determination value at point C may be set as an x coordinate value that is β % of the maximum value yₘₐₓ of the linearly approximated line within a range surrounding a positive point Pi shown in FIG. 12.

The first certainty-factor determination value at point C and the second certainty-factor determination value at point A may be set as a value larger than that at point B by a first set value and a value smaller than that at point B by a second set value with reference to the certainty-factor determination value at point B.

It is preferable that the aforementioned a and B or the aforementioned first set value and second set value are settable by the user, as appropriate.

In the actuation method of the image processing apparatus according to the second embodiment, the determination result of the first determination unit 23 and the determination result of the second determination unit 24 are not output if the special-light AI determination certainty factor is between the first certainty-factor determination value and the second certainty-factor determination value. Alternatively, for example, if the special-light AI determination certainty factor becomes smaller than the second certainty-factor determination value while the determination result of the first determination unit 23 is being output, switch may be made to an output of the determination result of the second determination unit 24. If the special-light AI determination certainty factor becomes greater than or equal to the first certainty-factor determination value while the determination result of the second determination unit 24 is being output, switch may be made to an output of the determination result of the first determination unit 23.

The horizontal axis (x axis) of the graph shown in each of FIG. 9 and FIG. 12 indicates the special-light AI determination certainty factor, but may alternatively indicate a white-light AI determination certainty factor. Similar to the calculation method of the special-light AI determination certainty factor, the white-light AI determination certainty factor can be calculated in accordance with Expression 1 or Expression 2. The probability (remission) and the probability (non-remission) in Expression 1 and Expression 2 are the determination probability of remission and the determination probability of non-remission, respectively, calculated from the determination result of the second determination unit 24.

The vertical axis (y axis) of the graph shown in each of FIG. 9 and FIG. 12 indicates the determination accuracy difference obtained by subtracting the WLI AI determination accuracy (accuracy_i_WLI) from the 410-nm AI determination accuracy (accuracy_i_410), but may alternatively be a determination accuracy difference obtained by subtracting the 410-nm AI determination accuracy (accuracy_i_410) from the WLI AI determination accuracy (accuracy_i_WLI).

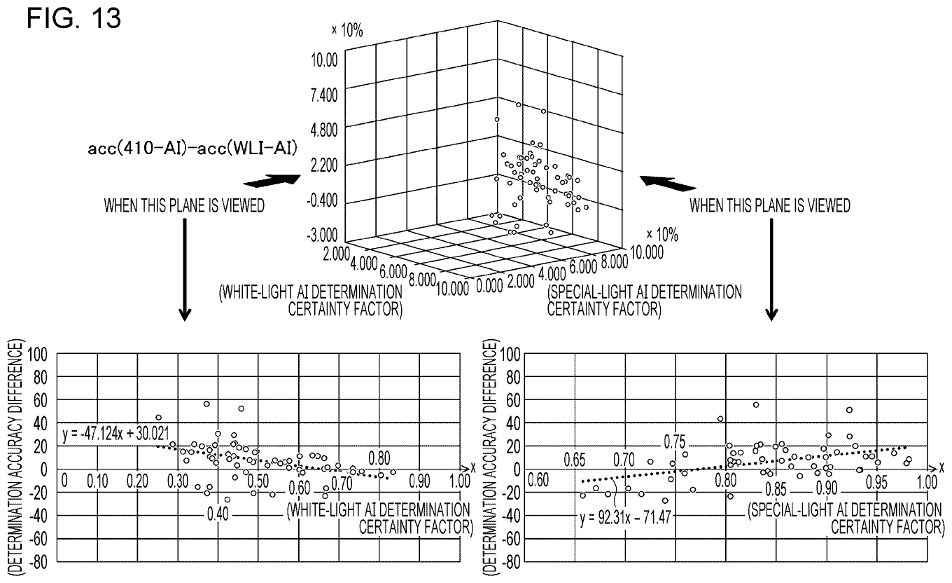

FIG. 13 illustrates other graphs related to the calculation method of the certainty-factor determination value.

In FIG. 13, the graph at the lower right side is the same as the graph shown in FIG. 12, whereas the graph at the lower left side is different from the graph at the lower right side in that the horizontal axis (x axis) uses the white-light AI determination certainty factor in place of the special-light AI determination certainty factor.

When a linearly approximated line is calculated from the graph at the lower left side, y=−47.124x+30.021. In this case, a point where the linearly approximated line intersects the x axis is 0.64, which is a value different from the certainty-factor determination value (0.77) at point B (see FIG. 12) calculated from the graph at the lower right side.

As indicated in the graph at the upper side in FIG. 13, it is preferable to set determination certainty factors as two axes of the special-light AI determination certainty factor and the white-light AI determination certainty factor, and to comprehensively decide whether to output either of the determination results of the first determination unit 23 and the second determination unit 24 or to select either of the special light source and the white light source based on the special-light AI determination certainty factor and the white-light AI determination certainty factor calculated during a diagnosis.

For example, the certainty-factor determination value of the special-light AI determination certainty factor and the certainty-factor determination value of the white-light AI determination certainty factor may be stored in the memory 27, the special-light AI determination certainty factor and the white-light AI determination certainty factor may be calculated during a diagnosis, the calculated special-light AI determination certainty factor and the calculated white-light AI determination certainty factor may be respectively compared with the two certainty-factor determination values stored in the memory 27, and it may be decided whether to output or not to output either of the determination results of the first determination unit 23 and the second determination unit 24.

### Others

As an alternative to the above embodiment in which a special light image and a white light image are alternately and successively acquired, either the first light source or the second light source may be automatically selected, and a first light source image or a second light source image (special light image, white light image) may be selectively acquired. For example, when the first light source image is selected, the determination certainty factor may be calculated from the determination result of the first determination unit to which the first light source image is input, and it may be decided whether either the first determination unit or the second determination unit is to be used (i.e., whether either the first light source or the second light source is to be selected) based on the calculated determination certainty factor and the certainty-factor determination value stored in the first memory. When the second light source image is selected, the determination certainty factor may be calculated from the determination result of the second determination unit to which the second light source image is input, and it may be decided whether either the first determination unit or the second determination unit is to be used (i.e., whether either the first light source or the second light source is to be selected) based on the calculated determination certainty factor and the certainty-factor determination value stored in the first memory.

The special light image may include, for example, two or more types of different special light images, such as a BLI image and an LCI image. In this case, it is necessary to prepare certainty-factor determination values and determination units respectively corresponding to the BLI image and the LCI image.

The hardware structure that executes various types of control of the image processing apparatus according to an exemplary embodiment of the invention includes various processors indicated below. The various processors include a central processing unit (CPU) as a general-purpose processor executing software (program) to function as one of various control units, a programmable logic device (PLD), such as a field-programmable gate array (FPGA), as a processor whose circuit configuration is changeable after being manufactured, and a dedicated electrical circuit, such as an application specific integrated circuit (ASIC), as a processor having a circuit configuration dedicatedly designed for executing a specific process.

One processing unit may be constituted of one of these various processors, or may be constituted of a combination of two or more of the processors of the same type or different types (e.g., a combination of multiple FPGAs or a combination of a CPU and an FPGA). Moreover, multiple control units may be constituted of a single processor. As a first example where multiple control units are constituted of a single processor, one processor may be constituted of a combination of at least one CPU and software, and the processor functions as multiple control units, as represented by, for example, client and server computers. A second example is a processor that realizes the function of an entire system including multiple control units by one integrated circuit (IC) chip, as represented by, for example, a system-on-a-chip (SoC). Accordingly, the various control units are constituted by using at least one of the aforementioned various processors as a hardware structure.

Exemplary embodiments of the invention are not limited to the above embodiments, and various modifications are possible so long as they do not depart from the spirit of the invention.

### REFERENCE SIGNS LIST

1 endoscope system
10 endoscope
20 processor apparatus
21 image acquiring unit
22, 200 processor
23 first determination unit
23A first learning model
24 second determination unit
24A second learning model
25 display control unit
26 input-output interface
27, 210 memory
28 operating unit
30 light source device
40 display device
100 special light image
102 white light image
110 image acquiring unit
112 determination-certainty calculating unit
114 decision unit
116 selecting unit
211 learning model
212 first learning data set
213 second learning data set
214 first parameter group
215 second parameter group
S10 to S70, S100 to S126 step

What is claimed is:

1. An image processing apparatus comprising:
a first determination unit and a second determination unit learned by using a first learning data set and a second learning data set obtained by photographing a target illuminated by a first light source and a second light source, respectively;
a first processor; and
a first memory storing a certainty-factor determination value,
wherein the first processor is configured to
acquire a first light source image obtained by photographing an evaluation target illuminated by the first light source and a second light source image obtained by photographing the evaluation target illuminated by the second light source,
input the first light source image to the first determination unit and calculate a determination certainty factor from a determination result of the first determination unit, and
decide whether to use the determination result of the first determination unit or to use a determination result of the second determination unit based on the determination certainty factor and the certainty-factor determination value stored in the first memory.

2. The image processing apparatus according to claim 1,
wherein the first processor outputs the determination result of the first determination unit when the determination certainty factor is greater than or equal to the certainty-factor determination value, and outputs the determination result of the second determination unit when the determination certainty factor is smaller than the certainty-factor determination value.

3. The image processing apparatus according to claim 1,
wherein the certainty-factor determination value stored in the first memory comprises a first certainty-factor determination value and a second certainty-factor determination value smaller than the first certainty-factor determination value, and
wherein the first processor outputs the determination result of the first determination unit when the determination certainty factor is greater than or equal to the first certainty-factor determination value, and outputs the determination result of the second determination unit when the determination certainty factor is smaller than the second certainty-factor determination value.

4. The image processing apparatus according to claim 1,
wherein, when the determination certainty factor is smaller than the certainty-factor determination value, the first processor acquires the second light source image in place of the first light source image, causes the second determination unit to receive the second light source image, and causes the second determination unit to output the determination result.

5. The image processing apparatus according to claim 1,
wherein the first processor is configured to
alternately and successively acquire the first light source image and the second light source image, and
select whether to output the first light source image to the first determination unit or to output the second light source image to the second determination unit based on the determination certainty factor and the certainty-factor determination value stored in the first memory.

6. The image processing apparatus according to claim 1,
wherein the determination result output by each of the first determination unit and the second determination unit indicates a presence or an absence of a lesion, a classification of the lesion, a degree of severity of the lesion, or remission or non-remission of an inflammatory disease.

7. The image processing apparatus according to claim 1,
wherein the learned first determination unit and the learned second determination unit comprise a learned first learning model and a learned second learning model that are learned by using the first learning data set and the second learning data set, respectively, and one or two second processors executing the first learning model and the second learning model.

8. The image processing apparatus according to claim 1,
wherein the first processor acquires a plurality of determination results with respect to a plurality of regions of the first light source image from the first determination unit, and calculates the determination certainty factor based on the plurality of determination results.

9. The image processing apparatus according to claim 1,
wherein the first processor acquires a plurality of determination results with respect to a plurality of successive first light source images from the first determination unit that successively receives the first light source images, and calculates the determination certainty factor based on the plurality of determination results.

10. The image processing apparatus according to claim 1,
wherein illumination light from one of the first light source and the second light source is special light in a narrow band, and illumination light from the other light source is white light, and
wherein each of the first light source image and the second light source image is an endoscopic image captured by an endoscope.

11. An actuation method of an image processing apparatus comprising a first determination unit and a second determination unit learned by using a first learning data set and a second learning data set obtained by photographing a target illuminated by a first light source and a second light source, respectively, a first processor, and a first memory storing a certainty-factor determination value, the actuation method of the image processing apparatus comprising:
a step for causing the first processor to acquire a first light source image obtained by photographing an evaluation target illuminated by the first light source and a second light source image obtained by photographing the evaluation target illuminated by the second light source;
a step for causing the first processor to input the first light source image to the first determination unit and calculate a determination certainty factor from a determination result of the first determination unit; and
a step for causing the first processor to decide whether to use the determination result of the first determination unit or to use a determination result of the second determination unit based on the determination certainty factor and the certainty-factor determination value stored in the first memory.

12. The actuation method of the image processing apparatus according to claim 11, further comprising:
a step for causing the first processor to output the determination result of the first determination unit when the determination certainty factor is greater than or equal to the certainty-factor determination value, and output the determination result of the second determination unit when the determination certainty factor is smaller than the certainty-factor determination value.

13. The actuation method of the image processing apparatus according to claim 11,
wherein the certainty-factor determination value stored in the first memory comprises a first certainty-factor determination value and a second certainty-factor determination value smaller than the first certainty-factor determination value, and
wherein the actuation method of the image processing apparatus further comprises:
a step for causing the first processor to output the determination result of the first determination unit when the determination certainty factor is greater than or equal to the first certainty-factor determination value, and output the determination result of the second determination unit when the determination certainty factor is smaller than the second certainty-factor determination value.

14. The actuation method of the image processing apparatus according to claim 11,
wherein the image processing apparatus further comprises:
a third processor and a second memory storing a plurality of first evaluation data sets and a plurality of second evaluation data sets obtained by photographing the target illuminated by the first light source and the second light source, respectively,
wherein the actuation method of the image processing apparatus further comprises:
a step for causing the third processor to input the first evaluation data sets and the second evaluation data sets respectively to the first determination unit and the second determination unit and acquire a plurality of first determination results and a plurality of second determination results from the first determination unit and the second determination unit;
a step for causing the third processor to calculate a plurality of first determination certainty factors from the plurality of first determination results;
a step for causing the third processor to calculate a plurality of first determination accuracies and a plurality of second determination accuracies from the plurality of first determination results and the plurality of second determination results, respectively; and
a step for causing the third processor to calculate the certainty-factor determination value based on a relationship between a determination accuracy difference and each first determination certainty factor, the determination accuracy difference indicating a difference between each first determination accuracy and each second determination accuracy at a same location of the target, and
wherein the first memory stores the calculated certainty-factor determination value.

15. The actuation method of the image processing apparatus according to claim 14,
wherein the third processor linearly approximates the relationship between the determination accuracy difference and each first determination certainty factor, and calculates, as the certainty-factor determination value, the first determination certainty factor when a sign of the determination accuracy difference on a linearly approximated line is reversed.
